(19) United States
(12) Patent Application Publication
(10) Pub. No.: US 2026/0132379 A1
(43) Pub. Date: May 14, 2026

(54) ISOLATION AND CULTURE CONDITIONS OF PERIPHERAL BLOOD-DERIVED STEM CELLS, AND INDUCTION OF DIFFERENTIATION INTO PROGENITOR CELLS BY USING SAME

(71) Applicant: SMART CELL LAB CO., LTD., Incheon (KR)

(72) Inventors: Jung Hwa Kang, Seoul (KR); Sung Hwan Hwang, Seoul (KR)

(21) Appl. No.: 19/107,218

(22) PCT Filed: Aug. 29, 2023
(86) PCT No.: PCT/KR2023/012770

§ 371 Date: Feb. 27, 2025

(30) Foreign Application Priority Data
Aug. 30, 2022 (KR) 10-2022-0108916

(51) Int. Cl. C12N 5/0775

(52) U.S. Cl. CPC C12N 5/0665; C12N 2501/11; C12N 2501/115; C12N 2501/15; C12N 2501/165; C12N 2501/24

(57) ABSTRACT

The objective of the present invention is to provide a method for effectively isolating and proliferating stem cells from the peripheral blood. A composition according to the present invention comprises interferon-gamma (IFN-γ) so as to increase the adhesion of stem cells isolated from the peripheral blood, and thus increases acquisition yield of the stem cells. Therefore, the composition and the peripheral blood-derived stem cell isolation and culture method, according to the present invention, are established and used to induce differentiation into progenitor cells, which can be differentiated into somatic cells of various target tissues, and are used for prevention or treatment of diseases.

### TECHNICAL FIELD

The present invention establishes conditions for effectively isolating and culturing stem cells present in peripheral blood and provides a composition for differentiation into progenitor cells capable of differentiating into somatic cells of various target tissues, and for treatment of diseases, using the stem cells.

### BACKGROUND ART

Stem cells may be obtained by isolating them from various tissues such as fat, bone marrow, and umbilical cord blood through surgical methods with the patient's consent.

In the case of adipose tissue extraction surgery, there is a possibility of serious side effects such as nerve or blood vessel destruction because the syringe tube is inserted into the adipose layer. Due to these side effects, patients and donors may have a psychological burden about the surgical procedures.

On the other hand, peripheral blood may be easily obtained by simply collecting blood without a surgical procedure.

Therefore, isolation of stem cells from peripheral blood requires no separate surgical procedure, and therefore it is convenient for patients and donors, and can be free from contamination problems that may occur during the surgical procedure. Therefore, it can provide an innovative approach to research and development of therapeutics using stem cells.

Therefore, the present invention provides a method of effectively isolating and culturing stem cells from peripheral blood.

In addition, the present invention provides a composition for differentiation into progenitor cells, which can be differentiated into somatic cells of various target tissues such as cartilage, myocardium, and nerves, and for treatment of diseases, using the peripheral blood-derived stem cells.

### DISCLOSURE

### Technical Problem

An object of the present invention is to provide a method of effectively isolating and proliferating stem cells from peripheral blood.

Another object of the present invention is to provide a medium composition for peripheral blood-derived stem cell attachment, including interferon-γ (INF-γ) and a growth factor, wherein the growth factor includes one or more growth factors selected from the group consisting of vascular endothelial growth factor (VEGF), transforming growth factor-beta (TGF-β), epidermal growth factor (EGF), and basic fibroblast growth factor (bFGF).

Still another object of the present invention is to provide a method of culturing peripheral blood-derived stem cells, including a step of isolating and culturing attached stem cells in a cell culture medium containing the medium composition for peripheral blood-derived stem cell attachment.

### Technical Solution

In order to achieve the above-described objects, the present invention provides a medium composition for peripheral blood-derived stem cell attachment, including interferon-gamma (IFN-γ) and a growth factor, wherein the growth factor includes at least one growth factor selected from the group consisting of vascular endothelial growth factor (VEGF), transforming growth factor-beta (TGF-β), epidermal growth factor (EGF), and basic fibroblast growth factor (bFGF).

In one embodiment, the present invention provides a medium composition for peripheral blood-derived stem cell attachment, the medium composition including: one or more substances selected from the group consisting of INF-γ, interferon-alpha (IFN-α), and tumor necrosis factor-alpha (TNF-α); and a growth factor.

In one embodiment, the medium composition used for peripheral blood-derived stem cell attachment of the present invention is not limited in its composition ratio, but the basic medium may include one or more substances selected from the group consisting of 1% to 20% of fetal bovine serum (FBS) and autologous plasma (auto serum) and may be a medium selected from the group consisting of Dulbecco's Modified Eagle's Medium (DMEM), Minimum Essential Media Eagle (MEM), Dulbecco's Modified Eagle Medium/Nutrient Mixture F-12 (DMEM/F-12), and α-MEM, but is not limited thereto.

The growth factor may include one or more growth factors selected from the group consisting of VEGF, TGF-β, EGF, and bFGF and preferably include EGF, FGF, and the like, but is not limited thereto.

When the IFN-γ is included in the medium composition capable of inducing the attachment of stem cells isolated from peripheral blood, the IFN-γ may be included in an amount of 1 to 200 ng/ml, preferably 1 to 50 ng/mL, based on the total weight of the medium composition, but is not limited thereto.

When the IFN-α is included in the medium composition capable of inducing the attachment of stem cells isolated from peripheral blood, the IFN-α may be included in an amount of 1 to 200 ng/mL, preferably 1 to 50 ng/mL, based on the total weight of the medium composition, but is not limited thereto.

When the TNF-α is included in the medium composition capable of inducing the attachment of stem cells isolated from peripheral blood, the TNF-α may be included in an amount of 1 to 50 ng/mL, preferably 1 to 10 ng/mL, based on the total weight of the medium composition, but is not limited thereto.

In the present invention, the medium containing IFN-γ for the attachment of stem cells isolated from peripheral blood may not include other factors such as growth factors other than IFN-γ.

In addition, the peripheral blood-derived stem cell may include a mesenchymal stem cell (MSC), but is not limited thereto.

The present invention provides a medium composition for peripheral blood-derived stem cell proliferation, the medium composition including: one or more substances selected from the group consisting of apo-transferrin, iron-dextran, and TNF-α; and a growth factor.

In one embodiment, the medium composition used for peripheral blood-derived stem cell proliferation of the present invention is not limited in its composition ratio, but the basic medium may include one or more substances selected from the group consisting of 1% to 20% of FBS and autologous plasma and may be a medium selected from the group consisting of DMEM, MEM, DMEM/F-12, and α-MEM, but is not limited thereto.

The growth factor may include one or more growth factors selected from the group consisting of VEGF, TGF-β, EGF, and bFGF and preferably include EGF, FGF, and the like, but is not limited thereto.

In the present invention, when the apo-transferrin is included in the medium composition capable of inducing the proliferation of isolated stem cells, the apo-transferrin may be included in an amount of 0.1 to 10 mg/mL, preferably 1 to 5 mg/mL, based on the total weight of the medium composition, but is not limited thereto.

When the iron-dextran is included in the medium composition capable of inducing the proliferation of isolated stem cells, the iron-dextran may be included in an amount of 1 to 10 μg/mL, preferably 1 to 5 μg/mL, based on the total weight of the medium composition, but is not limited thereto.

When the TNF-α is included in the medium composition capable of inducing the proliferation of isolated stem cells, the TNF-α may be included in an amount of 1 to 10 μg/mL, preferably 1 to 5 μg/mL, based on the total weight of the medium composition, but is not limited thereto.

In the present invention, the medium containing apo-transferrin and/or iron-dextran for proliferating stem cells isolated from peripheral blood may not contain other factors such as growth factors other than apo-transferrin and/or iron-dextran.

In addition, the peripheral blood-derived stem cell may include an MSC, but is not limited thereto.

In one embodiment, the present invention provides a method of culturing a stem cell isolated from peripheral blood in a cell culture medium containing a medium composition for peripheral blood-derived stem cell attachment including: one or more substances selected from the group consisting of IFN-γ, IFN-α, and TNF-α; and a growth factor; and a progenitor cell derived from a composition for inducing differentiation of the cell into a progenitor cell.

Specifically, the present invention provides a method of culturing a peripheral blood-derived stem cell, the method including the steps of: a) isolating and culturing attached stem cells in a cell culture medium containing a medium composition for peripheral blood-derived stem cell attachment; and b) culturing and proliferating the attached stem cells of a) in a cell culture medium containing a medium composition for peripheral blood-derived stem cell proliferation.

In one embodiment, a method of culturing a peripheral blood-derived stem cell is provided, the method including a step of isolating and culturing attached stem cells in a cell culture medium containing a medium composition for peripheral blood-derived stem cell attachment.

In one embodiment, as a result of isolating peripheral blood, attaching cells in a medium containing IFN-γ, and observing the cells, the degree of cell attachment was significantly different from that of the control group, and it was confirmed that the above cytokine was suitable for peripheral blood-derived stem cell attachment.

In one embodiment of the present invention, a useful biomarker may be LIN28, OCT4, NANOG, SOX2, CD29, CD90, and CD105, but is not limited thereto.

In the present specification, LIN28, OCT4, NANOG, SOX2 or CD90, and CD105 are marker genes or cell surface antigen markers indicating stem cells. OCT4, NANOG, and SOX2 are transcription factors essential for maintaining the phenotype of stem cells. LIN28 is a transcription factor that regulates the self-renewal of stem cells. CD29, also referred to as integrin beta-1 (ITGβ1), is a marker expressed in various cell types, including stem cells, and tissues such as blood and skin. CD90, also referred to as Thy-1, is a glycosylphosphatidylinositol (GPI)-anchored glycoprotein expressed in thymocytes, T cells, neurons, hematopoietic stem cells, and endothelial cells. CD105, also referred to as endoglin, is a type I membrane glycoprotein located on the cell surface that is part of TGF-β receptor complex and is used as a marker of hematopoietic cells.

In one embodiment, a chondroprogenitor cell induced from a composition containing one or more selected from the group consisting of ciprofloxacin and a growth factor for inducing differentiation from a peripheral blood-derived stem cell into a chondroprogenitor cell or a chondrocyte differentiated from a composition containing ciprofloxacin for inducing differentiation into a chondrocyte may be used for the prevention or treatment of diseases requiring cartilage regeneration, for example, cartilage-related diseases.

The cartilage-related disease may be, but is not limited to, one or more selected from the group consisting of osteoarthritis, arthritis deformans, chondrodystrophy, degenerative arthritis, rheumatoid arthritis, osteomalacia, fibrous osteitis, and achondroplasia.

The present invention also provides a method of preventing or treating a disease, the method including a step of administering a pharmaceutical composition to an individual in need thereof.

The pharmaceutical composition of the present invention may be applied in any dosage form, and more specifically, may be a parenteral dosage form. The parenteral dosage form may be in the form of an injection, an application, an aerosol, a patch, and the like.

Preparations for parenteral administration include sterile aqueous solutions, non-aqueous solvents, suspensions, emulsions, lyophilized preparations, and suppositories. As non-aqueous solvents and suspensions, propylene glycol, polyethylene glycol, vegetable oils such as olive oil, and injectable esters such as ethyl oleate may be used.

In order to formulate into an injectable formulation, the composition of the present invention may be mixed with a stabilizer or buffer in water to prepare a solution or suspension, which may be formulated into a unit dosage form in an ampoule or vial.

The term “stem cell” used in the present invention refers to a cell that has unlimited proliferation capacity (self-renewal capacity) in an undifferentiated state and the ability to differentiate into cells of various tissues (multi-differentiation potency) by a specific differentiation-inducing stimulus and may preferably be a mesenchymal stem cell (MSC).

The term “MSC” used in the present invention refers to a cell that has unlimited proliferation capacity in an undifferentiated state and the ability to differentiate into cells of various tissues by a specific differentiation-inducing stimulus. The MSC of the present invention may be any cell having differentiation capacity and proliferation capacity and may be derived from any animal, including humans, monkeys, pigs, horses, cows, sheep, dogs, cats, mice, and rabbits, preferably derived from humans, more preferably isolated from human adipose tissue, bone marrow, peripheral blood, or umbilical cord blood, and most preferably derived from peripheral blood.

The term “for attachment” used in the present invention refers to a composition that selects only stem cells included in the PBMCs attached to a culture dish after isolating a peripheral blood mononuclear cell (PBMC) from peripheral blood.

The term “for proliferation” used in the present invention refers to a composition capable of inducing the proliferation of the attached stem cells.

The term “progenitor cell” used in the present invention refers to a cell that has a superior ability to differentiate into a specific somatic cell than a stem cell, and the progenitor cell may be a chondroprogenitor cell, a cardiac progenitor cell, and a neural progenitor cell, but is not limited thereto.

The term “differentiation” used in the present invention refers to a phenomenon in which the structure or function of cells become specialized while the cells divide, proliferate, and grow, in other words, the form of function of cells, tissues, and the like of a living organism change to perform each given task. Differentiation of the present invention may be differentiation from a peripheral blood-derived stem cell into somatic cells including chondrocytes, cardiomyocytes, and neural cells, but is not limited thereto.

The term “individual” of the present invention includes an animal or human whose symptoms may be ameliorated by administration of the pharmaceutical composition according to the present invention, and preferably it may be a companion animal or a human, but is not limited thereto. By administering the prophylactic or therapeutic pharmaceutical composition according to the present invention to an individual, a disease can be effectively prevented or treated.

The term “administration” as used herein refers to introducing a predetermined substance into a human or animal by any appropriate method, and the administration route of the prophylactic or therapeutic composition according to the present invention may be oral or parenteral administration via any common route as long as it can reach the target tissue. In addition, the composition for preventing or treating cartilage-related diseases according to the present invention may be administered by any device through which an active ingredient may move to the target cell.

The preferred dosage of the pharmaceutical composition according to the present invention varies depending on the patient's conditions and weight, the severity of disease, drug form, and administration route and period, but may be appropriately selected by one of ordinary skill in the art.

### Advantageous Effects

The composition according to the present invention can increase the adhesiveness of stem cells isolated from peripheral blood by including one or more substances selected from the group consisting of interferon-gamma (IFN-γ), interferon-alpha (IFN-α), and tumor necrosis factor-alpha (TNF-α), thereby increasing the yield of obtained stem cells.

In addition, the composition according to the present invention can induce the proliferation of a peripheral blood-derived stem cell more rapidly than the existing culture method by including one or more substances selected from the group consisting of apo-transferrin, iron-dextran, and TNF-α.

Therefore, the composition and the method of isolating and culturing the peripheral blood-derived stem cells according to the present invention can be established and utilized to induce differentiation into progenitor cells capable of differentiating into somatic cells of various target tissues and can be used for the prevention or treatment of diseases.

### DESCRIPTION OF DRAWINGS

FIG. 1A shows the differences in attachment and proliferation of stem cells isolated from peripheral blood according to the presence or absence of interferon-gamma (IFN-γ) in the medium.

FIG. 1B shows the results of inducing cell proliferation in a medium containing TNF-α and apo-transferrin or iron-dextran.

FIG. 2 shows the basic characteristics of stem cells, illustrating positive reactions for CD29 and CD105 and negative reactions for CD34 in the immunophenotypes expressed by stem cells.

FIG. 3 shows the basic characteristics of stem cells, illustrating the levels of OCT4, LIN28, NANOG, and SOX2 marker genes expressed in mesenchymal stem cells.

FIG. 4A shows the results of differentiating peripheral blood-derived stem cells into chondrocytes and chondroprogenitor cells using ciprofloxacin and then confirming the differentiation by Alcian blue staining.

FIG. 4B shows the results of differentiating peripheral blood-derived stem cells into chondroprogenitor cells using ciprofloxacin and then confirming the expression of Col2a (type II collagen), ACAN (aggrecan), and DAPI (4′,6-diamidino-2-phenylindone), by immunofluorescence staining.

FIG. 4C shows the results of inducing the differentiation of mesenchymal stem cells into chondrocytes (somatic cells) and then confirming the expression of Col2a, aggrecan, and DAPI by immunofluorescence staining.

### MODES OF THE INVENTION

The advantages and features of the present invention and the method of achieving them will become clear with reference to the examples and manufacturing examples described in detail below. However, the present invention is not limited to the examples and manufacturing examples disclosed below, but may be implemented in different forms, and the examples and manufacturing examples are provided only to make the disclosure of the present invention complete and to fully inform one of ordinary skill in the art to which the present invention pertains of the scope of the invention.

### Example 1. Isolation and Culture of Peripheral Blood-Derived Stem Cells

Peripheral blood (blood donated from a hospital for non-profit patient treatment) was isolated using a density gradient with Ficoll at a ratio of 1:1. Thereafter, cells were cultured in Dulbecco's Modified Eagle Medium/Nutrient Mixture F-12 (DMEM/F-12) medium containing 1% to 20% auto serum or fetal bovine serum (FBS) to which 200 ng/mL interferon-gamma (IFN-γ) was added, to induce stem cell attachment.

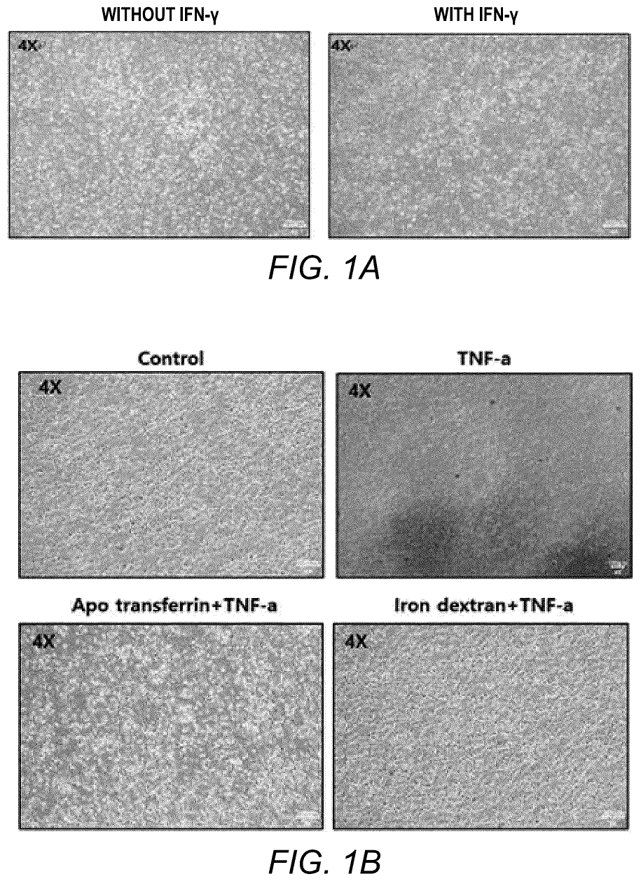

Thereafter, non-attached cells were removed, and then the medium was replaced with DMEM/F-12 medium containing 1% to 20% auto serum or FBS, and the cells were cultured until the stem cell density reached 80% (FIG. 1A).

### Example 2. Induction of Proliferation of Peripheral Blood-Derived Stem Cells

DMEM/F-12 medium containing 5 μg/mL TNF-α and 1% to 20% auto serum or FBS was mixed with 5 mg/mL apo-transferrin or 5 μg/mL iron-dextran. Peripheral blood-derived stem cells were cultured in the resulting medium to induce cell proliferation (FIG. 1B).

### Example 3. Confirmation of Surface Antigens on Peripheral Blood-Derived Stem Cells

Peripheral blood-derived stem cells with a cell density of 80% were washed with Dulbecco's phosphate buffered saline (DPBS) and recovered with TrypLE (Gibco, 12604-021). The cells were counted and suspended with a flow cytometry staining buffer (FACS buffer) at a concentration of 1×10⁵ cells/10 μL. The resulting cell suspension was placed in a 1.5 mL microcentrifuge tube and allowed to react for one hour at 4° C. using primary antibodies to CD29, CD105, or CD34 with fluorescent markers attached, while blocking light.

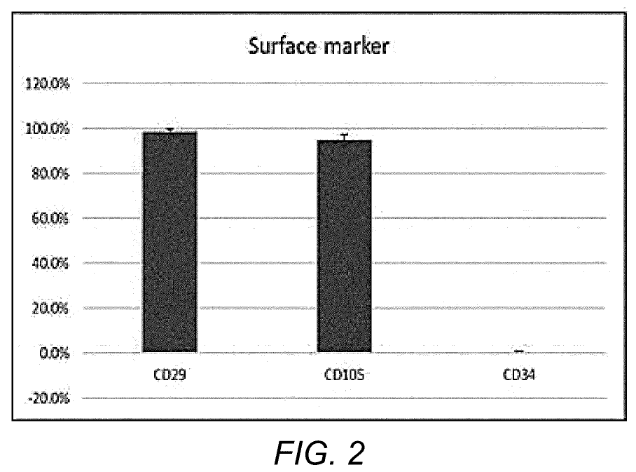

After completing the primary antibody reaction, the cells were washed three times with the FACS buffer, and then the antibody expression rate was confirmed using flow cytometry equipment. As a result, positive reactions for CD29 and CD105 and negative reactions for CD34 were confirmed (FIG. 2).

### Example 4. Confirmation Gene Levels of Peripheral Blood-Derived Stem Cells

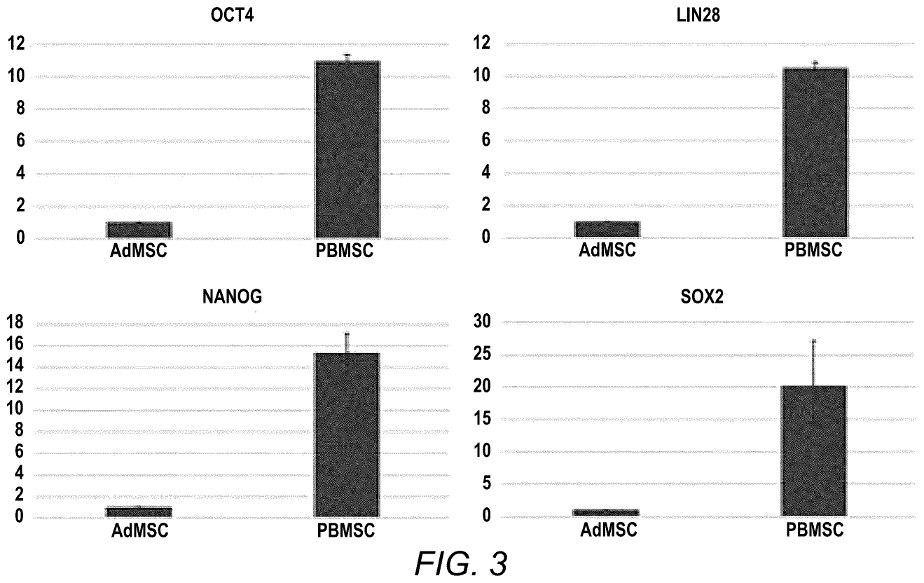

After 80% cell density was reached, RNA was extracted from peripheral blood-derived stem cells using Trizol, and then the expression of OCT4, NANOG, LIN28, and SOX2 genes was confirmed. As a result, the expression of stem cell-specific genes was confirmed (FIG. 3).

### Example 5. Confirmation of Differentiation of Peripheral Blood-Derived Stem Cells into Chondrocytes

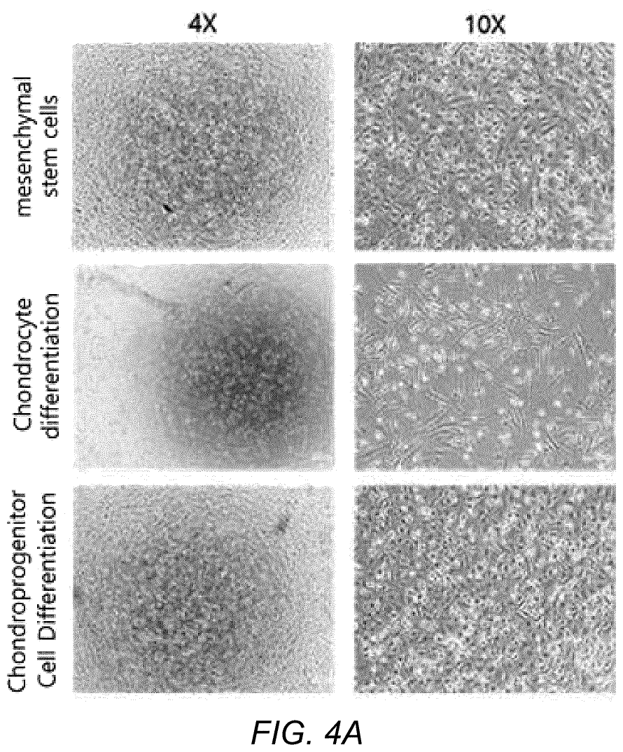

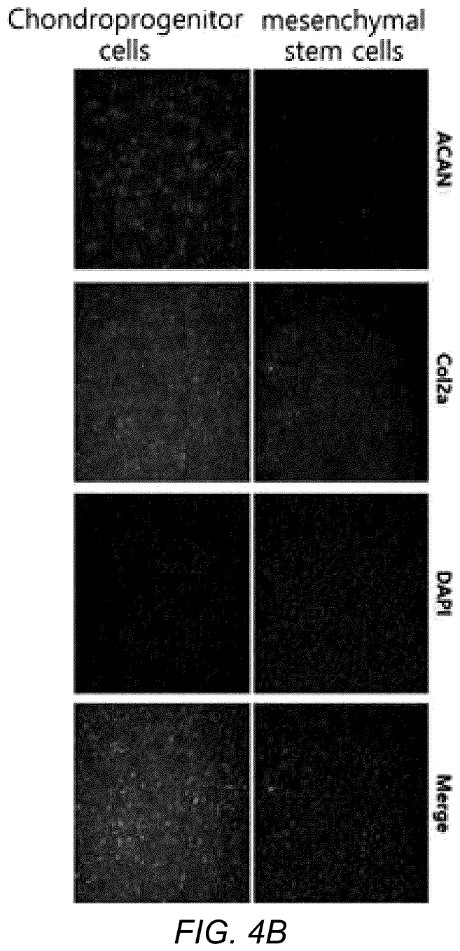

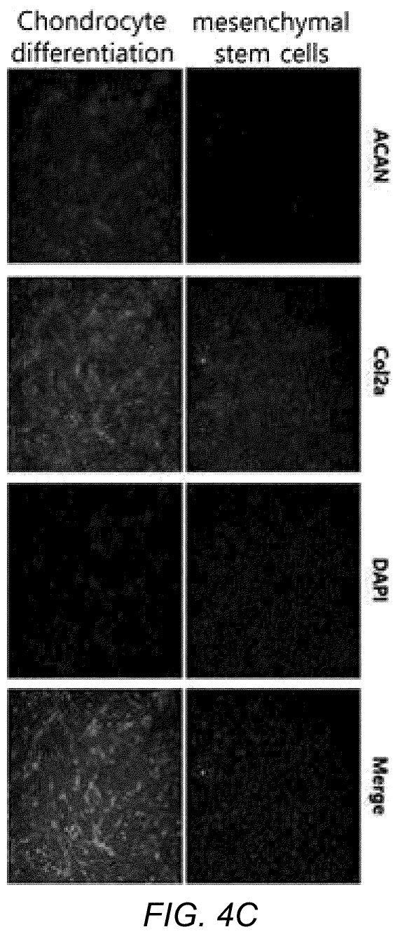

Peripheral blood-derived stem cells were induced to differentiate into chondrocytes for 16 days in a medium containing ciprofloxacin (FIG. 4A). The cells induced to differentiate for 16 days were washed with DPBS and fixed with 4% paraformaldehyde at room temperature for 30 minutes. The fixed cells were permeabilized with 0.05% Triton X-100 at room temperature for 10 minutes.

Thereafter, the cells were allowed to react with primary antibodies (Col2a and aggrecan) for one hour to one day and washed three times with DPBS. Thereafter, cells were allowed to react with secondary antibodies and washed three times with DPBS. Next, the nuclei were stained with DAPI for five minutes and washed three times with DPBS. The stained cells were mounted by adding the Vectashield mounting solution thereto in a dropwise manner. Finally, as a result of observing the sample with a confocal fluorescence microscope, it was confirmed that when the peripheral blood-derived stem cells are cultured in a medium containing ciprofloxacin, they may be differentiated into chondrocytes and chondroprogenitor cells (FIGS. 4B and 4C).

1. A medium composition for peripheral blood-derived stem cell attachment, comprising interferon-gamma (IFN-γ) and a growth factor, wherein the growth factor includes one or more growth factors selected from the group consisting of vascular endothelial growth factor (VEGF), transforming growth factor-beta (TGF-β), epidermal growth factor (EGF), and basic fibroblast growth factor (bFGF).

2. The composition of claim 1, wherein the medium is selected from the group consisting of Dulbecco's Modified Eagle's Medium (DMEM), Minimum Essential Media Eagle (MEM), Dulbecco's Modified Eagle Medium/Nutrient Mixture F-12 (DMEM/F-12), and α-MEM and contains one or more substances selected from the group consisting of fetal bovine serum (FBS) and autologous plasma (auto serum).

3. The composition of claim 1, wherein the IFN-γ is contained at a concentration of 1 to 200 ng/mL.

4. The composition of claim 1, wherein the peripheral blood-derived stem cell includes a mesenchymal stem cell.

5. The composition of claim 4, wherein the mesenchymal stem cell expresses CD29 and CD105.

6. A method of culturing a peripheral blood-derived stem cell, the method comprising steps of:
a) culturing a peripheral blood-derived cell in a cell culture medium containing the medium composition of claim 1 to attach a peripheral blood-derived stem cell; and
b) isolating and culturing the attached peripheral blood-derived stem cell.

7. A method of culturing a peripheral blood-derived stem cell, the method comprising steps of:
a) culturing a peripheral blood-derived cell in a cell culture medium containing the medium composition of claim 2 to attach a peripheral blood-derived stem cell; and
b) isolating and culturing the attached peripheral blood-derived stem cell.

8. A method of culturing a peripheral blood-derived stem cell, the method comprising steps of:
a) culturing a peripheral blood-derived cell in a cell culture medium containing the medium composition of claim 3 to attach a peripheral blood-derived stem cell; and
b) isolating and culturing the attached peripheral blood-derived stem cell.

9. A method of culturing a peripheral blood-derived stem cell, the method comprising steps of:
a) culturing a peripheral blood-derived cell in a cell culture medium containing the medium composition of claim 4 to attach a peripheral blood-derived stem cell; and
b) isolating and culturing the attached peripheral blood-derived stem cell.

10. A method of culturing a peripheral blood-derived stem cell, the method comprising steps of:
a) culturing a peripheral blood-derived cell in a cell culture medium containing the medium composition of claim 5 to attach a peripheral blood-derived stem cell; and
b) isolating and culturing the attached peripheral blood-derived stem cell.
